(19) United States
(12) Patent Application Publication
(10) Pub. No.: US 2018/0110740 A1
(43) Pub. Date: Apr. 26, 2018

(54) METHODS AND MATERIALS FOR TREATING ENDOMETRIOSIS

(71) Applicant: Mayo Foundation for Medical Education and Research, Rochester, MN (US)

(72) Inventor: Gaurang S. Daftary, Rochester, MN (US)

(73) Assignee: Mayo Foundation for Medical Education and Research, Rochester, MN (US)

(21) Appl. No.: 15/546,191

(22) PCT Filed: Jan. 26, 2016
(86) PCT No.: PCT/US2016/014806

§ 371 Date: Jul. 25, 2017

Related U.S. Application Data
(60) Provisional application No. 62/108,438, filed on Jan. 27, 2015.

(51) Int. Cl. A61K 31/122; A61P 15/00; A61K 31/60; A61K 31/426; A61K 31/09

(52) U.S. Cl. CPC A61K 31/122; A61P 15/00; A61K 31/09; A61K 31/426; A61K 31/60

(57) ABSTRACT

This document provides methods and materials for treating endometriosis. For example, methods and materials for using histone acetyl transferase inhibitors (e.g., garcinol) to treat endometriosis are provided.

### CROSS REFERENCE TO RELATED APPLICATIONS

This application claims the benefit of U.S. Provisional Ser. No. 62/108,438 filed Jan. 27, 2015. This disclosure of the prior application is considered part of (and is incorporated by reference in) the disclosure of this application.

### BACKGROUND

### 1. Technical Field

This document relates to methods and materials involved in treating endometriosis. For example, this document provides methods and materials for using histone acetyl transferase inhibitors (e.g., garcinol) to treat endometriosis.

### 2. Background Information

Endometriosis affects about 10 percent of reproductive age women. It can cause pain, infertility, and sexual dysfunction. Although altering sex-steroid levels with or without surgical excision of lesions is the mainstay treatment, it can be unfocused and is often unsatisfactory.

### SUMMARY

This document provides methods and materials for treating endometriosis. For example, this document provides methods and materials for using histone acetyl transferase inhibitors (e.g., garcinol) to treat endometriosis. As described herein, administering a histone acetyl transferase inhibitor such as garcinol to a female mammal with endometriosis can result in reduced scarring from endometriosis or reduced progression of scarring from endometriosis. In some cases, having the ability to reduce scarring from endometriosis or to slow progression of scarring from endometriosis can allow patients with endometriosis to experience less pain.

In general, one aspect of this document features a method for slowing progression of endometriosis in a female mammal. The method comprises, or consists essentially of, (a) identifying a female mammal as having endometriosis, and (b) administering a histone acetyl transferase inhibitor to the female mammal, thereby slowing progression of endometriosis within the female mammal. The female mammal can be a human. The histone acetyl transferase inhibitor can be selected from the group consisting of garcinol, anacardic acid, CPTH-2, curcurmin, and MB-3.

In another aspect, this document features a method for slowing development of endometriosis in a female mammal. The method comprises, or consists essentially of, (a) identifying a female mammal as being at risk for developing endometriosis, and (b) administering a histone acetyl transferase inhibitor to the female mammal, thereby slowing development of the endometriosis within the female mammal. The female mammal can be a human. The histone acetyl transferase inhibitor can be selected from the group consisting of garcinol, anacardic acid, CPTH-2, curcurmin, and MB-3.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 (top panel) is a schematic representation of a KLF11 polypeptide binding to the COL1A1 promoter. FIG. 1 (middle panel) is a photograph of a chromatin immunoprecipitation (ChIP) analysis using human endometrial stromal cells (HESC). COL1A1 and COL1A2 elements were amplified from input DNA prior to immune precipitation (Input). COL1A1 and COL1A2 elements also were amplified when an anti-KLF11 antibody, but not when a control IgG antibody, was used in the chromatin immunoprecipitation reaction. FIG. 1 (bottom panel) is a graph plotting relative luciferase levels for cells transfected with either wild type KLF11 or corresponding empty vector (EV). KLF11 repressed COL1A1 and 1A2 expression in contrast to EV. *=p<0.05.

FIG. 2 (top panel) is a schematic representation of a Sin3 polypeptide/KLF11 polypeptide complex binding to the COL1A1 promoter. FIG. 2 (bottom panel) is a graph plotting relative luciferase levels for cells transfected with either wild type KLF11, corresponding empty vector (EV), or KLF11EAPP a mutant wherein amino acids E29 and A30 were mutagenized to proline residues. This mutation prevents SIN3/HDAC binding to KLF11. Whereas KLF11 repressed COL1A1 expression in contrast to EV, the repression was reversed with the KLF11EAPP mutant, indicating that KLF11 recruited SIN3/HDAC to repress COL1A1 expression. *=p<0.05.

FIGS. 3A, 3B, 3E, and 3F are photographs of mouse tissue with the white arrows pointing to the lesions. FIGS. 3A-D, I: Klf11^{-/-} animals exhibited larger lesions compared to wild type macroscopically as well as microscopically (100× magnification). FIGS. 3E-H: Prolific scarring also was present in Klf11^{-/-} animals in contrast to wild type animals. FIGS. 3C, 3D, 3G, and 3H are photographs of mouse tissue sections stained with masson trichrome to represent collagen. COL1A1 mRNA expression was increased 8-fold in implants from Klf11^{-/-} animals (FIG. 3J) compared wild type animals (*=p<0.05).

FIG. 4 is a schematic diagram showing the design of the mouse endometriosis model. Two 0.5 cm uterine fragments are everted, so that the endometrium faces the peritonel cavity. These are then sutured to the flank peritoneum on either side by 8/0 proline microfilament suture.

FIG. 5 contains photographs of Klf11^{-/-} and wt animals induced to produce endometriosis lesions and treated with DMSO (control), garcinol (0.2 μg/g of body weight, or suberoyl anilide hydroxamic acid (SAHA; 50 μg/g of body weight). SAHA is also known as N-hydroxy-N′-phenyl-octanediamide or vorinostat. Endometriotic progression was evaluated three weeks after treatment. The arrows point to and define lesion associated scarring.

### DETAILED DESCRIPTION

This document provides methods and materials involved in treating endometriosis. For example, this document provides methods and materials for using histone acetyl transferase inhibitors (e.g., garcinol) to treat endometriosis, to reduce the progression of endometriosis, to reduce the development of endometriosis, or to slow the onset of endometriosis within a female mammal.

Any type of female mammal having endometriosis or at risk for developing endometriosis can be treated as described herein. For example, female humans and other primates such as monkeys having endometriosis can be treated with one or more histone acetyl transferase inhibitors. In some cases, dogs, cats, horses, cows, pigs, sheep, mice, and rats can be treated with one or more histone acetyl transferase inhibitors as described herein.

Any appropriate method can be used to identify a mammal having endometriosis or as being at risk for developing endometriosis. For example, laparoscopy, biopsy, and pathology techniques can be used to identify a human having endometriosis.

Once identified as having endometriosis or as being at risk for developing endometriosis, the mammal can be administered or instructed to self-administer one or more histone acetyl transferase inhibitors. Examples of histone acetyl transferase inhibitors include, without limitation, garcinol, anacardic acid, CPTH2, curcurmin, and MB-3.

In some cases, one or more histone acetyl transferase inhibitors (e.g., one, two, three, four, five, or more histone acetyl transferase inhibitors) can be administered to a female mammal to reduce scarring from endometriosis. For example, two or more histone acetyl transferase inhibitors having diverging therapeutic properties can be administered to a female mammal to reduce scarring from endometriosis. In some cases, one or more histone acetyl transferase inhibitors can be formulated into a pharmaceutically acceptable composition for administration to a mammal having endometriosis or as being at risk for developing endometriosis. For example, a therapeutically effective amount of garcinol can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more histone acetyl transferase inhibitors can be designed for oral or parenteral (including subcutaneous, intramuscular, intravenous, and intradermal) administration. When being administered orally, a pharmaceutical composition containing one or more histone acetyl transferase inhibitors can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

Such injection solutions can be in the form, for example, of a sterile injectable aqueous or oleaginous suspension. This suspension may be formulated using, for example, suitable dispersing or wetting agents (such as, for example, Tween 80) and suspending agents. The sterile injectable preparation can be a sterile injectable solution or suspension in a non-toxic parenterally-acceptable diluent or solvent, for example, as a solution in 1, 3-butanediol. Examples of acceptable vehicles and solvents that can be used include, without limitation, mannitol, water, Ringer's solution, and isotonic sodium chloride solution. In addition, sterile, fixed oils can be used as a solvent or suspending medium. In some cases, a bland fixed oil can be used such as synthetic mono- or di-glycerides. Fatty acids, such as oleic acid and its glyceride derivatives can be used in the preparation of injectables, as can natural pharmaceutically-acceptable oils, such as olive oil or castor oil, including those in their polyoxyethylated versions. In some cases, these oil solutions or suspensions can contain a long-chain alcohol diluent or dispersant.

In some cases, a pharmaceutically acceptable composition including one or more histone acetyl transferase inhibitors can be administered locally or systemically. For example, a composition containing a histone acetyl transferase inhibitor can be administered locally by injection into lesions at surgery or by subcutaneous administration of a sustained release formulation. In some cases, a composition containing a histone acetyl transferase inhibitor can be administered systemically orally or by injection to a mammal (e.g., a human).

Effective doses can vary depending on the severity of the endometriosis, the route of administration, the age and general health condition of the subject, excipient usage, the possibility of co-usage with other therapeutic treatments such as use of other agents, and the judgment of the treating physician.

An effective amount of a composition containing one or more histone acetyl transferase inhibitors can be any amount that reduces the severity of a symptom of a condition being treated (e.g., endometriosis) without producing significant toxicity to the mammal. For example, an effective amount of a histone acetyl transferase inhibitor such as garcinol can be from about 10 mg/kg to about 100 mg/kg (e.g., from about 50 mg/kg to about 75 mg/kg). In some cases, between about 650 mg and about 6500 mg of a histone acetyl transferase inhibitor such as garcinol can be administered to an average sized female human (e.g., about 65 kg human) daily for about four to about eight weeks (e.g., about five to six weeks). If a particular mammal fails to respond to a particular amount, then the amount of histone acetyl transferase inhibitor can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., endometriosis) may require an increase or decrease in the actual effective amount administered.

The frequency of administration can be any frequency that reduces the severity of a symptom of a condition to be treated (e.g., endometriosis) without producing significant toxicity to the mammal. For example, the frequency of administration can be from about once a week to about three times a day, or from about twice a month to about six times a day, or from about twice a week to about once a day. The frequency of administration can remain constant or can be variable during the duration of treatment. A course of treatment with a composition containing one or more histone acetyl transferase inhibitors can include rest periods. For example, a composition containing one or more histone acetyl transferase inhibitors can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., endometriosis) may require an increase or decrease in administration frequency.

An effective duration for administering a composition containing one or more histone acetyl transferase inhibitors can be any duration that reduces the severity of a symptom of the condition to be treated (e.g., endometriosis) without producing significant toxicity to the mammal. Thus, the effective duration can vary from several days to several weeks, months, or years. In general, the effective duration for the treatment of endometriosis can range in duration from six months to one year. Multiple factors can influence the actual effective duration used for a particular treatment. For example, an effective duration can vary with the frequency of administration, effective amount, use of multiple treatment agents, route of administration, and severity of the condition being treated.

In certain instances, a course of treatment and the severity of one or more symptoms related to the condition being treated (e.g., endometriosis) can be monitored. Any appropriate method can be used to determine whether or not the severity of a symptom is reduced. For example, the severity of a symptom of endometriosis (e.g., scarring) can be assessed using office laparoscopy at different time points. The invention will be further described in the following examples, which do not limit the scope of the invention described in the claims.

### EXAMPLES

### Example 1

### Epigenetic Therapies Arrest Progression of Endometriosis in the Klf1^{-/-} Disease Model for Endometriosis

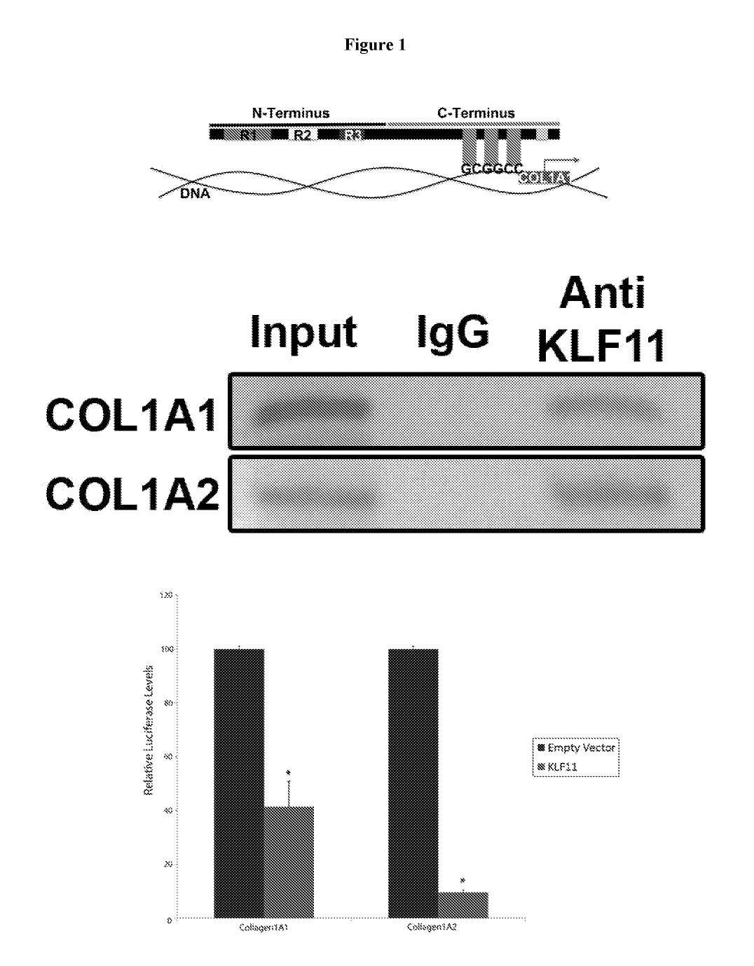

KLF11-mediated transcriptional regulation of COL1A1 was assessed by chromatin immunoprecipitation (ChIP), luciferase, immunohistochemistry, and qPCR assays in human endometrial stromal (HESC) cells. To evaluate KLF11 binding to COL1 promoter elements, ChIP was performed using HESC. Both COL1A1 and 1A2 elements were amplified from input DNA prior to immune precipitation (FIG. 1, middle panel). These elements also were amplified when an anti-KLF11 antibody, but not when a control IgG antibody, was used in a prior immunoprecipitation reaction (FIG. 1, middle panel). These results demonstrate that KLF11 directly binds the COL1 regulatory regions and regulates its transcription and thus expression.

In addition, luciferase assay was performed to assess the interaction between KLF11 polypeptides and the collagen 1A1 and 1A2 promoter elements. Briefly, collagen 1A1 and 1A2 promoter activity was assessed using reporter assays designed to express luciferase when either of the collagen 1A1 and 1A2 promoter elements are active. HESC transfected with a vector designed to express wild type-KLF11 polypeptides exhibited a reduction in collagen 1A1 and 1A2 promoter activity as compared to that observed in control HESC transfected with an empty vector (FIG. 1, bottom panel). These results demonstrate that KLF11 is a transcriptional repressor of COL1A1 and COL1A2 expression (FIG. 1, top panel).

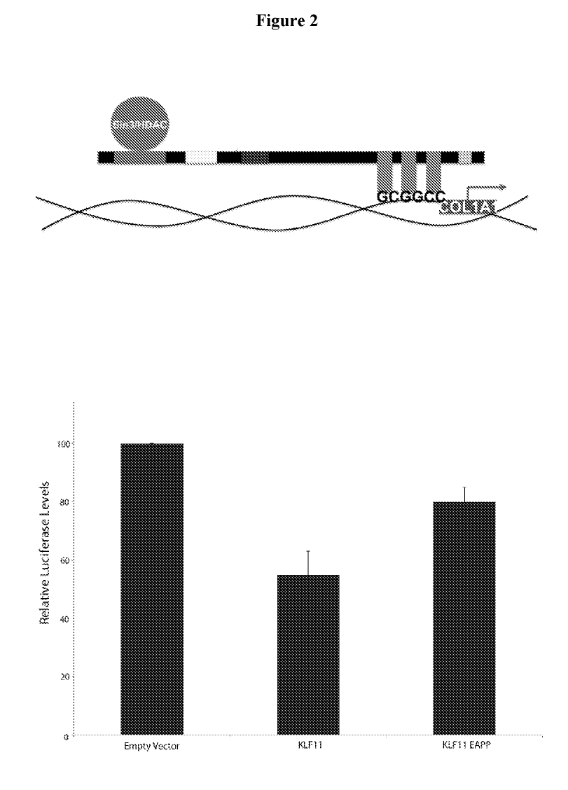

In another experiment, KLF11 was mutagenized to abrogate SIN3/HDAC recruitment (FIG. 2, top panel). Briefly, mutations were introduced into the KLF11 Sin3 binding domain, where the E19 and A20 positions were each mutated to a proline residue to produce KLF11 EAPP. KLF11 EAPP is unable to bind and recruit the Sin3/Hdac complex. In the absence of Sin3/HDAC recruitment, the repression of COL1A1 promoter activity was reversed (FIG. 2, bottom panel), likely from non-deacetylation of COL1A1 promoter histones.

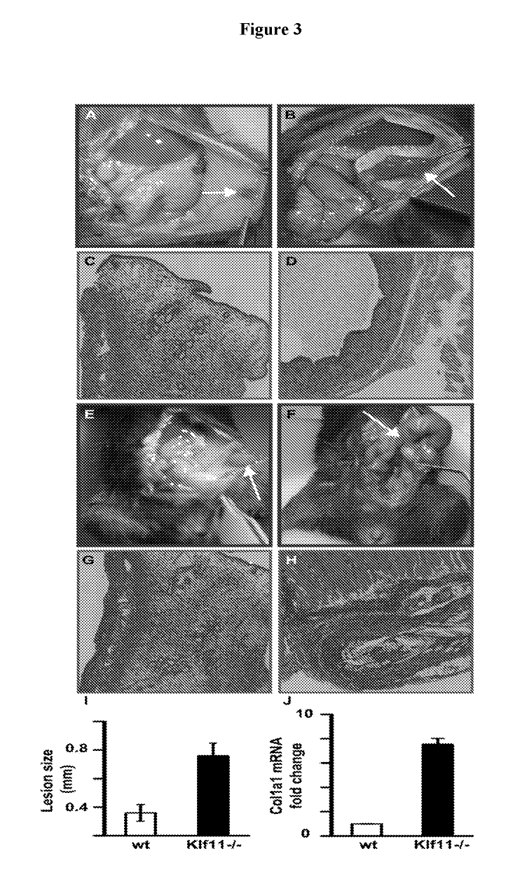

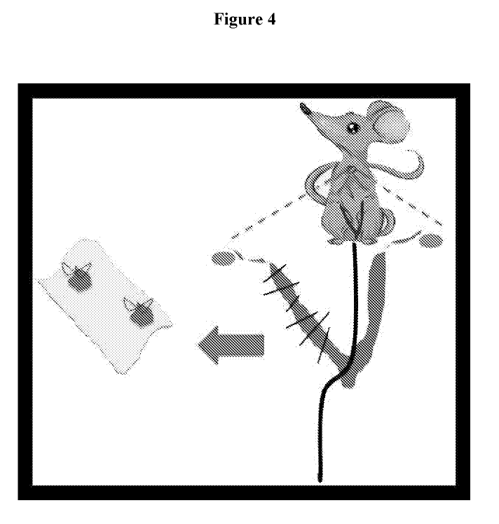

In another experiment, endometriotic lesions were induced in Klf11^{-/-} or wildtype (wt) mice (n=10 for each group) by implanting autologous 5 mm uterine segments onto the peritoneum (FIGS. 3 and 4). Lesion growth and prolific scarring were observed in Klf11^{-/-} animals (FIGS. 3B and 3F). In contrast, wt animals exhibited minimal adhesions and observable lesion regression (FIGS. 3A and 3E; p<0.05). In addition, COL1A1 polypeptide expression was increased in implants from Klf11^{-/-} animals (FIGS. 3D and 3H) as compared to those from wt animals (FIGS. 3C and 3G). Further, lesion size was larger in Klf11^{-/-} animals than in wt animals (FIG. 3I), and Klf11^{-/-} animals exhibited elevated COL1A1 mRNA expression as compared to wt animals (FIG. 3J). These results demonstrate that Klf11 has a role in preventing progression of endometriotic lesions.

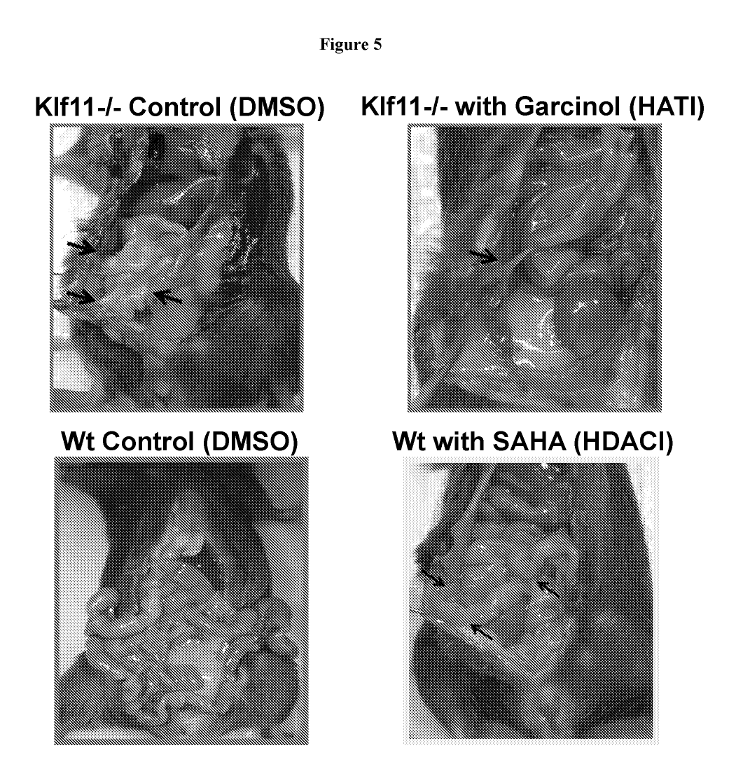

A similar endometriotic lesion experiment was performed using Klf11^{-/-} and wt animals that were treated with either DMSO (vehicle), garcinol (0.2 μg/g of body weight, or SAHA (50 μg/g of body weight). Endometriotic progression was evaluated three weeks after treatment. Prevention of COL1 promoter histone acetylation using the histone acetyl transferase inhibitor garcinol in Klf11^{-/-} mice ameliorated scarring (FIG. 5). In addition, in wt animals, antagonism of Klf11/Hdac by the histone deacetylase inhibitor SAHA resulted in an induction of de novo fibrosis (FIG. 5). These results demonstrate that histone acetyl transferase inhibitors such as garcinol can be used to treat endometriosis.

Taken together, the results provided herein demonstrate that COL1 promoter activity is regulated by KLF11 via a novel epigenetic pathway and that therapeutic alteration of endometriotic progression can be achieved using histone acetyl transferase inhibitors such as garcinol.

### Example 2

### Epigenetic Therapy: Arrest of Environmental Dioxin Induced Endometriotic Progression

Diversity in etiopathogenesis of endometriosis results in heterogeneity and recalcitrance to therapy. Treatments targeting molecular dysregulation in individual patients are needed. Exposure to common environmental contaminant 2,3,7,8-tetrachlorodibenzodioxin (Dioxin) increases incidence and progression of endometriosis. Dioxin ubiquitously activates cytochrome (CYP450) enzymes via the aryl hydrocarbon pathway to alter metabolism and disease proclivity. KLF11, a transcription factor implicated in endometriosis, represses CYP450 enzymes via an epigenetic Histone Deacetylase (HDAC) mechanism. A translationally relevant epigenetic mechanism is characterized that targets environmentally induced endometriosis.

Ishikawa cells were treated with Dioxin (0.1-10 nM) and KLF11 or EV-control. Treatment effects were determined on CYP3A4, a well-characterized CYP450 gene that is also an endometrial estrogen metabolizing enzyme. Promoter and gene expression were determined by reporter assay, qPCR, and western blot. CYP3A4 activity was determined by a specific substrate assay. Promoter histone code was evaluated by ChIP in Ishikawa treated with Dioxin and Histone Acetyl Transferase Inhibitor (HATI) or controls. The Dioxin-endometriosis model was created per published protocols in wild type mice gavaged with Dioxin (3 μg/kg) or vehicle. Animals were additionally treated 0.2 μg/gm/day of HATI or vehicle control (N=10/grp). Disease progression was evaluated in 12 weeks by morphometry, IHC, and qPCR.

CYP3A4 was dose-dependently activated by Dioxin. KLF11 antagonized activation of CYP3A4 promoter, expression and function. KLF11 recruited HDAC to repress CYP3A4. Congruently, in Dioxin-only treated mice, prolific endometriotic progression accompanied lesional epithelial CYP3A4 activation. In contrast, in mice treated with Dioxin+HATI, significantly diminished disease progression was associated with lower CYP3A4 expression (Score 26.4 vs. 15.5 in Dioxin and Dioxin+HATI groups, respectively; P<0.05). HATI antagonized Dioxin by specific deacetylation of the CYP3A4 promoter.

The metabolic profile of any tissue impacts its role in health and disease. Dioxin ubiquitously targets CYP450 enzymes with multi-system effects. The results provided herein demonstrate that a translationally relevant KLF11/HDAC mechanism antagonizes Dioxin. The effects of environmental low dose Dioxin exposure can therefore be mitigated by this translationally relevant antagonistic epigenetic pathway.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for slowing progression of endometriosis in a female mammal, wherein said method comprises:
(a) identifying a female mammal as having endometriosis, and
(b) administering a histone acetyl transferase inhibitor to said female mammal, thereby slowing progression of endometriosis within said female mammal.

2. The method of claim 1, wherein said female mammal is a human.

3. The method of claim 1, wherein said histone acetyl transferase inhibitor is selected from the group consisting of garcinol, anacardic acid, CPTH-2, curcurmin, and MB-3.

4. A method for slowing development of endometriosis in a female mammal, wherein said method comprises:
(a) identifying a female mammal as being at risk for developing endometriosis, and
(b) administering a histone acetyl transferase inhibitor to said female mammal, thereby slowing development of said endometriosis within said female mammal.

5. The method of claim 4, wherein said female mammal is a human.

6. The method of claim 4, wherein said histone acetyl transferase inhibitor is selected from the group consisting of garcinol, anacardic acid, CPTH-2, curcurmin, and MB-3.
